(19) United States
(12) Patent Application Publication
(10) Pub. No.: US 2008/0242991 A1
(43) Pub. Date: Oct. 2, 2008

(54) Probe for Ultrasound Diagnosis and Ultrasound Diagnostic System Using the Same

(75) Inventors: Chang Ho Moon, Seoul (KR); Won Soon Hwang, Seoul (KR); Jeong Cheol Seo, Gyeonggi-do (KR)

Correspondence Address: JONES DAY, 222 EAST 41ST ST, NEW YORK, NY, 10017, US

(73) Assignee: MEDISON CO., LTD., Kangwon-do, Korea (KP)

(21) Appl. No.: 12/088,391

(22) PCT Filed: Sep. 27, 2006
(86) PCT No.: PCT/KR2006/003846

§ 371 Date: Mar. 27, 2008

(30) Foreign Application Priority Data
Sep. 27, 2005 (KR) 10-2005-0089648
Sep. 29, 2005 (KR) 10-2005-0091067

(51) Int. Cl. A61B 8/14

(52) U.S. Cl. 600/445; 600/459

(57) ABSTRACT

The present invention relates to a probe, which is adapted to match acoustic impedances and shield undesired signals, and an ultrasound diagnostic system configured to use such a probe. The probe for conducting ultrasound diagnosis includes a transducer array for transmitting ultrasound signals to a target object and receiving the ultrasound signals reflected from the target object. The probe of the present invention also includes multiple membranes for covering the transducer array, wherein the multiple membranes include at least two membranes formed from different materials and having different thicknesses.

### TECHNICAL FIELD

The present invention generally relates to a probe for conducting ultrasound diagnosis and an ultrasound diagnostic system using the same, and more particularly to a probe adapted to match acoustic impedances and shield undesired signals and an ultrasound diagnostic system using the same.

### BACKGROUND ART

An ultrasound diagnostic system has become an important and popular diagnostic tool due to its wide range of applications. Specifically, due to its non-invasive and non-destructive nature, the ultrasound diagnostic system has been extensively used in the medical profession. Modern high-performance ultrasound diagnostic systems and techniques are commonly used to produce two or three-dimensional (2D or 3D) diagnostic images of a target object.

The ultrasound diagnostic system generally uses a probe, which includes a transducer array having a plurality of transducers, to transmit and receive ultrasound signals. The ultrasound diagnostic system forms ultrasound images of the internal structures of the target object by electrically exciting the transducer to generate ultrasound pulses that travel into the target object. The ultrasound pulses produce ultrasound echoes since they are reflected from a discontinuous surface of acoustic impedance of the internal structure, which appears as discontinuities to the propagating ultrasound pulses. The various ultrasound echoes return to the transducer and are converted into electrical signals, which are amplified and processed to produce ultrasound data for an image of the internal structure of the target object.

A typical ultrasound probe includes a transducer array and other mechanical hardware such as a probe housing, potting material and electrical shielding. The transducer array is typically produced by stacking various layers in sequence.

Conventionally, the probe employs a single membrane made from rubber or plastic in order to form its housing. However, ultrasound signals scanned by such a probe may be distorted or include noises or undesired signals, which can hinder accurate diagnosis. Thus, the probe needs to be improved in order to obtain high-sensitivity and high-resolution 3D ultrasound images.

### DISCLOSURE OF INVENTION

### Technical Problem

In order to solve the above-mentioned problems, the present invention provides a probe adapted to match acoustic impedances and shield undesired signals without distorting or losing ultrasound signals and an ultrasound diagnostic system configured to use such a probe.

### Technical Solution

In accordance with an aspect of the present invention, there is provided a probe for conducting ultrasound diagnosis, which includes: a transducer array for transmitting ultrasound signals to a target object and receiving the ultrasound signals from the target object; and multiple membranes for covering the transducer array, wherein the multiple membranes includes at least two membranes formed from different materials and having different thicknesses.

In accordance with another aspect of the present invention, there is provided an ultrasound diagnostic system, including: a probe for scanning ultrasound signals; a processor for producing an ultrasound image based on the ultrasound signals provided from the probe; and a display unit for displaying the produced ultrasound image, wherein the probe includes a transducer array for transmitting the ultrasound signals to a target object and receiving the ultrasound signals from the target object and the multiple membranes for covering the transducer array, and wherein the multiple membranes include at least two membranes formed from different materials and having different thicknesses.

### ADVANTAGEOUS EFFECTS

In accordance with the present invention, multiple membranes can minimize a difference in the acoustic impedances between the transducer array and the membrane such that high-sensitivity and high-resolution 3D ultrasound images can be formed. Further, the membranes including a shielding layer made from an electrically conductive material are applied to the probe so as to shield noises or undesired signals such that high-sensitivity and high-resolution 3D ultrasound images can be formed.

### BRIEF DESCRIPTION OF THE DRAWINGS

FIG. 1 schematically shows a cross-sectional view of a probe with multiple membranes for performing ultrasound diagnosis in accordance with one embodiment of the present invention.

FIG. 2 is a schematic diagram showing a shielding layer inserted into the multiple membranes.

FIG. 3 is a schematic diagram showing a shielding layer formed on a transducer array-facing surface of the multiple membranes.

FIG. 4 schematically shows an ultrasound diagnostic system configured to use the probe shown in FIG. 1.

### BEST MODE FOR CARRYING OUT THE INVENTION

Hereinafter, one embodiment of the present invention will be described with reference to the accompanying drawings.

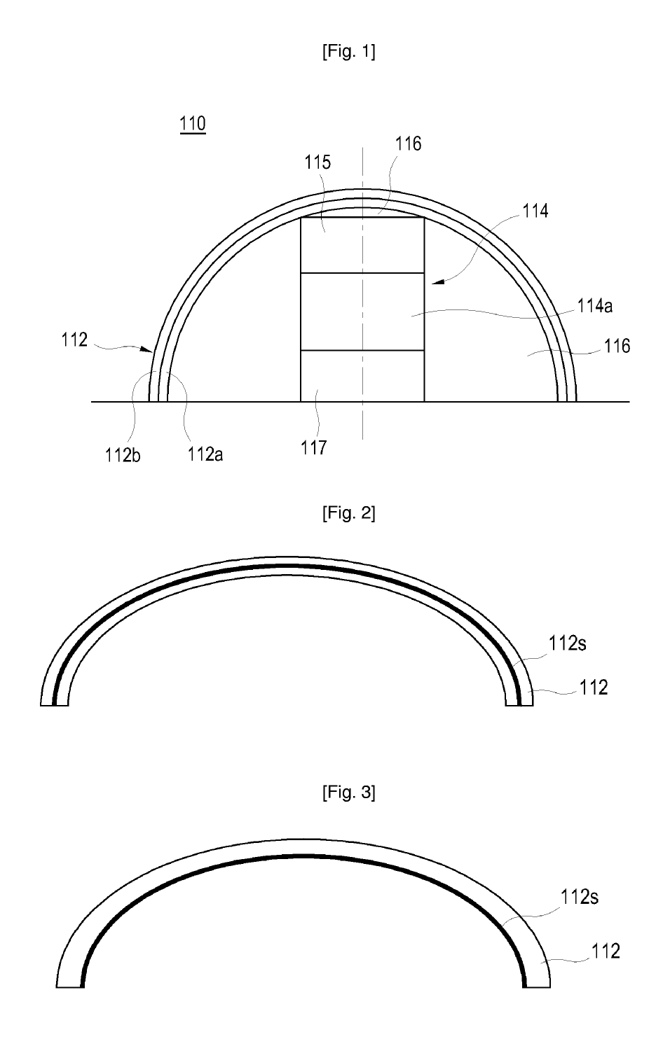

FIG. 1 schematically shows a cross-sectional view of a probe for performing ultrasound diagnosis in accordance with one embodiment of the present invention. The probe of the present invention includes multiple membranes.

Referring to FIG. 1, the probe 110 includes multiple membranes 112 and a transducer array 114 for transmitting and receiving ultrasound signals. Oil may be provided in the space between the membrane 112 and the transducer array 114 so as to facilitate the operations of the probe 110. The membrane 112 serves as a cover for protecting the transducer array 114. The transducer array 114 is formed with piezoelectric elements 114a. Further, the probe 110 includes a matching layer 115 covering the piezoelectric elements 114a, a lens 116 covering the matching layer 115 and a backing layer 117 supporting the piezoelectric elements 114a. Although the probe 110 includes the matching layer 115 in this embodiment, the multiple membranes 112 can serve as a matching layer. In this case, the matching layer 115 can be omitted.

The multiple membranes 112 include an inner membrane 112a and an outer membrane 112b, which are formed from different materials and have different thicknesses. The inner membrane 112a and the outer membrane 112b face the transducer array 114 and the target object, respectively. When ultrasound passes through the multiple membranes 112, the ultrasound velocity in the inner membrane 112a is different from that in the outer membrane 112b. It is preferable that the thicknesses of the inner and outer membranes 112a and 112b are formed to be substantially one fourth of the ultrasound wavelengths in the inner and outer membranes 112a and 112b, respectively, to minimize the difference between the acoustic impedances. Further, due to high acoustic impedance of the transducer 114 and low acoustic impedance of the body, it is preferable that an impedance value of the inner membrane 112a is larger than an impedance value of the outer membrane 112b in order to reduce acoustic reflection (loss).

The multiple membranes 112 include two membranes (i.e., inner and outer membranes) in this embodiment. However, it should be noted that the multiple membranes 112 can include one or more other membranes in addition to the inner and outer membranes. Also, when using three or more membranes, the thickness of each membrane is preferably formed to be substantially one fourth of an ultrasound wavelength in each membrane. Further, an impedance value of an inner layer is preferably larger than an impedance value of an outer layer.

FIG. 2 is a schematic diagram showing a shielding layer inserted into the multiple membranes. In FIG. 2, the shielding layer 112s is disposed inside the membrane 112. However, as shown in FIG. 3, the shielding layer 112s may be formed on a surface of the inner membrane 112a facing the transducer array 114. Either membrane 112a or 112b can serve as a shielding layer.

The shielding layer 112s contains an electrically conductive element selected from the group consisting of Ag, Cu, Au, Al, Mg, Zn, Pt, Fe and Pb. For example, the shielding layer 112s can be formed by sputtering.

The shielding layer 112s prevents the undesired signals or noises in signals passing through the membrane 112 from invading the transducer array 114. That is, the shielding layer 112s allows only the acoustic signals to pass through the multiple membranes 112 to enable the formation of high-sensitivity and high-resolution ultrasound images.

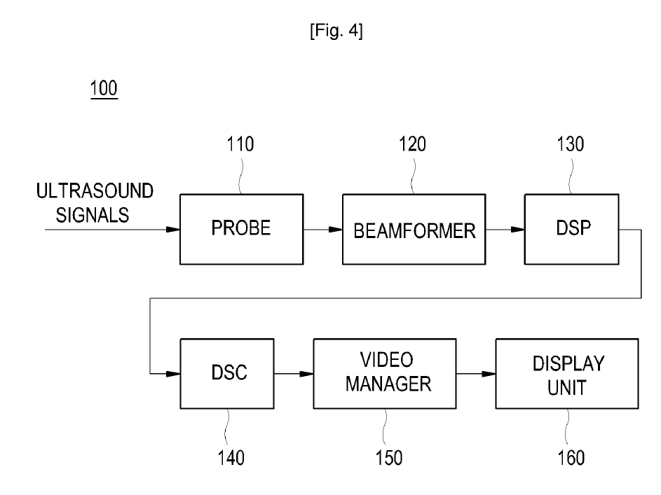

FIG. 4 schematically shows an ultrasound diagnostic system configured to adopt and use the probe shown in FIG. 1. Referring to FIG. 4, the ultrasound diagnostic system 100 includes a probe 110, a beamformer 120, a digital signal processor (DSP) 130, a digital scan converter (DSC) 140, a video manager 150 and a display unit 160. The beamformer 120, the DSP 130, the DSC 140 and the video manager 150 may be formed with a single processor, which produces ultrasound images based on ultrasound signals provided by the probe 110.

As shown in FIG. 1, the probe 110 includes the transducer array 114 for transmitting and receiving the ultrasound signals. The probe 110 also includes the multiple membranes 112, which serve as a housing for covering the transducer array 114. The multiple membranes 112 include at least two membranes, for example, an inner membrane 112a and an outer membrane 112b, which are formed from different materials and have different thicknesses. When ultrasound passes through the multiple membranes 112, the ultrasound velocity in the inner membrane 112a is different from that in the outer membrane 112b. Preferably, the thicknesses of the inner and outer membranes 112a and 112b are formed to be substantially one fourth of the ultrasound wavelengths in the inner and outer membranes 112a and 112b, respectively, to minimize the difference between the acoustic impedances.

Further, a shielding layer may be disposed between the membranes or formed on a surface of the inner membrane 112a facing the transducer array 114. It should be noted herein that any one of the inner and outer membranes 112a and 112b can be used as a shielding layer. The shielding layer contains an electrically conductive element selected from the group consisting of Ag, Cu, Au, Al, Mg, Zn, Pt, Fe and Pb. The shielding layer prevents the undesired signals or noises in signals passing through the membrane from invading the transducer array 114. That is, the shielding layer allows only acoustic signals to pass through the multiple membranes 112.

The beamformer 120 produces frame data signals by focusing ultrasound echo signals received by the transducer array. The DSP 130 produces ultrasound image data from the frame data signals by digital signal processing. The DSC 140 converts the ultrasound image data into scan-converted data in the X-Y format for video display.

The video manager 150 converts the scan-converted frame data into appropriate video signals for use in the display unit 160. The display unit 112 displays an ultrasound image produced based on the video signals sent from the video manager 150 so as to provide it to a user.

While the present invention has been described and illustrated with respect to a preferred embodiment of the invention, it will be apparent to those skilled in the art that variations and modifications are possible without deviating from the broad principles and teachings of the present invention which should be limited solely by the scope of the claims appended hereto.

### INDUSTRIAL APPLICABILITY

Multiple membranes in accordance with the present invention can minimize a difference in the acoustic impedances between the transducer array and the membrane. Further, the membranes including a shielding layer made from an electrically conductive material are applied to the probe so as to shield noises or undesired signals. Thus, high-sensitivity and high-resolution 3D ultrasound images can be formed by using the multiple membranes.

1. A probe for conducting ultrasound diagnosis, comprising:
a transducer array for transmitting ultrasound signals to a target object and receiving the ultrasound signals reflected from the target object; and
multiple membranes for covering the transducer array,
wherein the multiple membranes include at least two membranes formed from different materials and having different thicknesses.

2. The probe of claim 1, wherein a thickness of each membrane is formed to be substantially one fourth of an ultrasound wavelength in the each membrane.

3. The probe of claim 1, wherein the multiple membranes include an inner membrane facing the transducer array and an outer membrane facing the target object, and wherein an impedance value of the inner membrane is larger than an impedance value of the outer membrane.

4. The probe of claim 1, wherein the multiple membranes are a matching layer.

5. The probe of claim 1, wherein one of the multiple membranes is a shielding layer.

6. The probe of claim 5, wherein the shielding layer contains an electrically conductive element.

7. The probe of claim 6, wherein the electrically conductive element is selected from a group consisting of Ag, Cu, Au, Al, Mg, Zn, Pt, Fe and Pb.

8. An ultrasound diagnostic system, comprising:
a probe for scanning ultrasound signals;
a processor for producing an ultrasound image based on the ultrasound signals provided by the probe; and
a display unit for displaying the produced ultrasound image,
wherein the probe includes a transducer array for transmitting the ultrasound signals to a target object and receiving the ultrasound signals reflected from the target object and the multiple membranes for covering the transducer array, and
wherein the multiple membranes include at least two membranes formed from different materials and having different thicknesses.

9. The ultrasound diagnostic system of claim 8, wherein a thickness of each membrane is formed to be substantially one fourth of an ultrasound wavelength in the each membrane.

10. The ultrasound diagnostic system of claim 8, wherein the multiple membranes include an inner membrane facing the transducer array and an outer membrane facing the target object, and wherein an impedance value of the inner membrane is larger than an impedance value of the outer membrane.

11. The ultrasound diagnostic system of claim 8, wherein the multiple membranes are a matching layer.

12. The ultrasound diagnostic system of claim 8, wherein one of the multiple membranes is a shielding layer.

13. The ultrasound diagnostic system of claim 12, wherein the shielding layer contains an electrically conductive element.

14. The ultrasound diagnostic system of claim 13, wherein the electrically conductive element is selected from a group consisting of Ag, Cu, Au, Al, Mg, Zn, Pt, Fe and Pb.
